(19) United States
(12) Patent Application Publication
(10) Pub. No.: US 2025/0230473 A1
(43) Pub. Date: Jul. 17, 2025

(54) TRANSGENIC STRAIN FOR PRODUCING 3-HYDROXY-3-METHYLBUTYRIC ACID AND PRODUCTION METHOD OF 3-HYDROXY-3-METHYLBUTYRIC ACID

(71) Applicant: National Yang Ming Chiao Tung University, HSINCHU (TW)

(72) Inventors: I-Ethan Lan, HSINCHU (TW); Jan-Chi Huang, HSINCHU (TW); Jian-Dong Lai, HSINCHU (TW); Chia-Yen Hung, HSINCHU (TW)

(21) Appl. No.: 18/632,342

(22) Filed: Apr. 11, 2024

(30) Foreign Application Priority Data
Jan. 12, 2024 (TW) 113101406

(51) Int. Cl. C12P 7/52; C12N 9/10; C12N 9/16; C12N 9/88; C12N 15/52

(52) U.S. Cl. CPC C12P 7/52; C12N 9/1025; C12N 9/1029; C12N 9/16; C12N 9/88; C12N 15/52; C12Y 203/01009; C12Y 203/0301; C12Y 301/02; C12Y 401/01; C12Y 402/01018

(57) ABSTRACT

A transgenic strain for producing 3-hydroxy-3-methylbutyric acid is provided. The transgenic strain comprises a host cell with an anabolic pathway for converting carbon sources into acetyl-CoA and a plurality of exogenous genes. These exogenous genes comprise genes encoding acetyl-CoA acetyltransferase (AtoB) or acetoacetyl-CoA thiolase (NphT7), and genes encoding hydroxymethylglutaryl-CoA synthetase (MvaS), a gene encoding 3-hydroxy-3-methylglutaconyl-CoA dehydratase (LiuC), a gene encoding 3-methylglutaryl-CoA decarboxylase (AibAB), a gene encoding thioester hydrolase (YciA, TesB, MenI or YqiA). A method for producing 3-hydroxy-3-methylbutyric acid using the above genetically modified strain is also provided.

### CROSS-REFERENCE TO RELATED APPLICATION

This application claims the priority benefit of Taiwan application serial no. 113101406, filed Jan. 12, 2024, the full disclosure of which is incorporated herein by reference.

### REFERENCE TO SEQUENCE LISTING

This application includes a Sequence Listing filed electronically as an XML file named “Sequence Listing_PROFN-24004-USPT.xml”, created on Apr. 10, 2024, with a size of 15,808 bytes. The Sequence Listing is incorporated herein by reference.

### BACKGROUND

### Technical Field

The disclosure relates to a method for producing 3-hydroxy-3-methylbutyrate. More particularly, the disclosure relates to a method for producing 3-hydroxy-3-methylbutyrate using microorganisms.

### Description of Related Art

Currently, there are two main methods for producing 3-hydroxy-3-methylbutyric acid (HMB): chemical synthesis and biosynthesis.

In the chemical synthesis method, a typical approach involves using diacetone alcohol and oxidizing agents as raw materials. After acidification and organic solvent extraction, the resulting product reacts with calcium hydroxide or calcium chloride to form salts of 3-hydroxy-3-methylbutyric acid. This method yields approximately 50 kg of product from the reaction of about 80 kg of diacetone alcohol and 1200 kg of 10% sodium hypochlorite, with the product containing 98.7 wt % 3-hydroxy-3-methylbutyric acid. Thus, the conversion rate of diacetone alcohol in this reaction is approximately 61%. Subsequently, 250 kg of ethanol is added, followed by treatment with calcium hydroxide to obtain 95 kg of solid calcium salt of HMB. However, this chemical synthesis method faces several challenges, including stringent reaction conditions requiring precise control of temperature with high heat and low-temperature environments, which demands sophisticated equipment and personnel. The oxidizing agents used are corrosive, and the resulting liquid contains high salt content, necessitating extensive organic solvent extraction. The conversion efficiency of the synthesis reaction to form salts with calcium is typically below 62%, and there is a high dependency on petrochemical raw materials.

To address the challenges of the chemical synthesis method, some research teams have begun investigating biosynthesis, which mainly includes enzymatic and microbial conversion pathways. One application of enzymatic biosynthesis involves the use of crude protein from cells of Galactomyces reessii, which contains hydratase enzymes. This method employs the precursor 3-methylbutyric acid for hydration, resulting in the production of 3-hydroxy-3-methylbutyric acid. However, this approach has drawbacks, including the high cost and large quantity requirement for purifying hydratase from mycelium, as well as difficulties in maintaining the enzymatic reaction's activity. Therefore, developing a more feasible method that utilizes renewable resources as carbon sources and employs microorganisms as metabolic factories for HMB production is desirable.

Currently, some research teams utilize Escherichia coli as a host to construct the production pathway of HMB and conduct fermentation using glucose as the carbon source. However, the yield of this method is not yet satisfactory. Additionally, there are efforts using Yarrowia lipolytica as a host to construct production pathways, also employing glucose as the microbial carbon source for fermentation. However, the main culture medium in this method typically relies on complex media, often requiring the addition of yeast extract or casein tryptone, further increasing the cultivation costs.

### SUMMARY

In one aspect, the present invention is directed to a transgenic strain for producing 3-hydroxy-3-methylbutyric acid. The transgenic strain comprises a host cell and a plurality of exogenous genes. The host cell has an anabolic pathway for converting a carbon source into acetyl-CoA, wherein the host cell is Escherichia coli, cyanobacteria, or yeast, and the carbon source comprises at least one of glucose, xylose, acetic acid or acetate, methanol, and carbon dioxide. The exogenous genes are located in the host cell to assemble an anabolic pathway from acetyl-CoA (Ac-COA) to 3-hydroxy-3-methylbutyrate (HMB). The exogenous genes comprise a first, second, third, fourth, and fifth exogenous genes. The first exogenous gene is a gene encoding acetyl-CoA acetyltransferase (AtoB) or acetyl-CoA thiolase (NphT7), or a gene with 80% or more sequence identity to SEQ ID NO: 1 or SEQ ID NO: 2 and possessing AtoB activity or NphT7 activity. The second exogenous gene is a gene encoding hydroxymethylglutaryl-CoA synthase (MvaS), or a gene with 70% or more sequence identity to SEQ ID NO: 3 and possessing MvaS activity. The third exogenous gene is a gene encoding 3-hydroxy-3-methylglutaryl CoA dehydratase (LiuC), or gene with 50% or more sequence identity to SEQ ID NO: 4 and possessing LiuC activity. The fourth exogenous gene is a gene encoding 3-methylglutaryl-CoA decarboxylase (AibAB), or a gene with 70% or more sequence identity to SEQ ID NO: 5 and SEQ ID NO: 6 and possessing AibAB activity when expressed and combined. The fifth exogenous gene is a gene encoding a thioester hydrolase (YciA, TesB, MenI, or YqiA), or a gene with 60% or more sequence identity to SEQ ID NO: 7, SEQ ID NO: 8, SEQ ID NO: 9, or SEQ ID NO: 10, and possessing YciA, TesB, MenI, or YqiA activity when expressed.

According to an embodiment of this invention, the transgenic strain further comprises at least a recombinant plasmid carrying the exogenous genes.

According to another embodiment of this invention, the exogenous genes are integrated into a genome of the host cell.

According to another embodiment of this invention, the Escherichia coli comprises a plurality of substrains directly utilizing exogenous carbon sources to synthesize acetyl-CoA in vitro, and the Escherichia coli substrains comprise BW25113, XL1-Blue, DH5a, W3110, NEBExpress®, Rosetta (DE3), W strain, crooks, and the probiotic E. coli Nissle 1917.

According to another embodiment of this invention, the Escherichia coli has gene deletions of metabolic pathways competing with the anabolic pathway of 3-hydroxy-3-methylbutyratethe.

According to another embodiment of this invention, a gene of the Escherichia coli encoding pyruvate oxidase (PoxB) is knockout to reduce the production of acetate byproducts in the pathway synthesizing acetate from pyruvate.

According to another embodiment of this invention, a gene of the Escherichia coli encoding citrate synthase (GltA) in the TCA cycle is knockout to increase the yield of 3-hydroxy-3-methylbutyrate as the main product.

According to yet another embodiment of this invention, the cyanobacteria comprise Synechococcus elongatus.

According to yet another embodiment of this invention, the yeast comprises Pichia pastoris.

In another aspect, this invention is directed to a method for the production of 3-hydroxy-3-methylbutyrate. The method comprises culturing the transgenic strains as described above to allow the transgenic strain to produce 3-hydroxy-3-methylbutyrate. The culturing conditions comprise a first culturing temperature before inducing the expression of the exogenous genes in the transgenic strain is 37±2° C., a second culturing temperature after inducing the expression of the exogenous genes in the transgenic strain is 30±2° C., and a dissolved oxygen level in a bacterial solution of the transgenic strain is 30%.

According to an embodiment of this invention, a culture medium used is M9 minimal medium when the transgenic strain is the Escherichia coli.

According to another embodiment of this invention, a culture medium used is modified BG-11 medium containing 50±10 mM NaHCO₃ when the transgenic strain is the cyanobacteria.

According to yet another embodiment of this invention, a culture medium used is YPB complex medium when the transgenic strain is the yeast.

From the above embodiments of the present invention, it can be seen that compared to the current chemical methods using petroleum derivatives as raw materials for producing HMB, the embodiments of the present invention enable microorganisms to directly utilize organic carbon sources (such as glucose, glycerol, xylose, etc.) for HMB production. This not only reduces the reliance on petrochemical raw materials in the HMB market but also reduces the generation of harmful byproducts. Furthermore, there is currently no literature or patents proving that the same HMB anabolic pathway can be expressed in Escherichia coli, Synechococcus elongatus, or Pichia pastoris as in the embodiments described above, and serve as metabolic factories for HMB production. Especially, Escherichia coli can achieve high yields of HMB with just the minimal medium M9 with pure salts, showing significant potential for industrial application and development.

The foregoing presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

In order to make the above and other aspects, features, advantages, and embodiments of the present invention more apparent and understandable, the description of the accompanying drawings is as follows.

FIGS. 1A-1B show the designed synthetic metabolic pathway of 3-hydroxy-3-methylbutyrate within microorganisms.

FIG. 2A illustrates a schematic diagram of establishing a dual-plasmid system for the synthetic metabolic pathway of 3-hydroxy-3-methylbutyrate in Escherichia coli.

FIG. 2B depicts the screening of various homologous genes from the ASKA clone library in Escherichia coli, capable of effectively enhancing the production of HMB in reaction step 106 of FIG. 1B. It also illustrates the concentrations of HMB and acetate in the bacterial solution after 24 hours of fermentation.

FIG. 3 illustrates a schematic diagram of the Escherichia coli dual-plasmid expression system utilizing the second plasmid to carry other microbial homologous genes, along with the concentrations of metabolites after 24 hours of fermentation.

FIGS. 4A-4C respectively depict the comparison of protein sites of LiuC, AibA, and AibB from strains Stigmatella aurantiaca DW4/3-1 (SEQ ID NO: 4-6) and Myxococcus xanthus DK 1622 (SEQ ID NO: 11-13), as used in Chinese patent CN107075536A.

FIG. 5 illustrates the establishment of a single-plasmid system in Escherichia coli for expressing the HMB synthesis pathway and replacing the species sources of the genes liuC, aibA, and aibB. It compares the production and yield of HMB and the main byproduct acetate (AC) after 24 hours of fermentation in various strains.

FIG. 6 depicts the production of HMB and the byproduct acetate (AC) after 24 hours of fermentation in different substrains of Escherichia coli expressing the HMB synthesis pathway.

FIG. 7 illustrates the production of HMB and the byproduct acetate (AC) after 24 hours of fermentation in Escherichia coli cultured using complex media or synthetic media.

FIG. 8A depicts a schematic diagram of culturing Escherichia coli using various common carbon sources to obtain HMB through different metabolic pathways, along with the theoretical yield of HMB achievable using different carbon sources.

FIG. 8B illustrates the production and yield of HMB after 24 hours of fermentation in Escherichia coli cultured using different single carbon sources.

FIG. 9A depicts the time course of glucose consumption during the large-scale cultivation of transgenic Escherichia coli for HMB production in a bioreactor.

FIG. 9B illustrates the time course of HMB and the main byproduct acetate (AC) production in transgenic Escherichia coli cultivated in a bioreactor for large-scale production.

FIG. 10A illustrates a schematic diagram of competitive metabolic pathways for the production of HMB.

FIG. 10B shows the comparison of the 24-hour production of HMB and the main byproduct acetate (AC) in Escherichia coli producing HMB with knocking out genes from competitive pathways.

FIG. 11A illustrates a schematic diagram of the plasmid containing the complete HMB anabolic pathway genes, with NSII (Neutral site II) from the photosynthetic autotrophic cyanobacterium Synechococcus elongatus as the recombinant site.

FIG. 11B shows the time course of HMB production in the Synechococcus elongatus RM24 strain cultivated in shake flasks for 16 days.

FIG. 12A illustrates a schematic diagram of the plasmid containing the complete HMB anabolic pathway genes, with NSI (Neutral site I) from the photosynthetic autotrophic cyanobacterium Synechococcus elongatus as the recombinant site.

FIG. 12B shows the time course of HMB production in the Synechococcus elongatus RM37 strain cultivated in shake flasks for 9 days.

FIG. 13A illustrates a schematic diagram of the expression vector for producing HMB in the eukaryotic organism Pichia pastoris as the host.

FIG. 13B shows the HMB production in wild-type and genetically engineered Pichia pastoris strains cultivated in YNB basic medium for 72-120 hours.

FIG. 13C illustrates the HMB production from wild-type and genetically engineered Pichia pastoris strains cultivated in YPB complex medium for 72-120 hours.

### DETAILED DESCRIPTION

Based on the above, a transgenic strain and a method for producing 3-hydroxy-3-methylbutyric acid (HMB) will be provided. In the following description, exemplary microorganisms and exemplary production methods for producing HMB will be introduced.

To provide a comprehensive description of the embodiments of the present invention, corresponding figures will be included below, along with explanatory descriptions of various aspects and specific embodiments of the invention. However, not all embodiments of the present invention require these technical details. That is, the implementation or application of the present invention is not limited to any particular form but may involve combining features and method steps from multiple embodiments.

### Designing an Anabolic Pathway of 3-Hydroxy-3-Methylbutyric Acid (HMB) Within Microorganisms

First, it is necessary to design a synthetic metabolic pathway for 3-hydroxy-3-methylbutyric acid (HMB) within microorganisms, enabling the use of microorganisms as production tools for HMB.

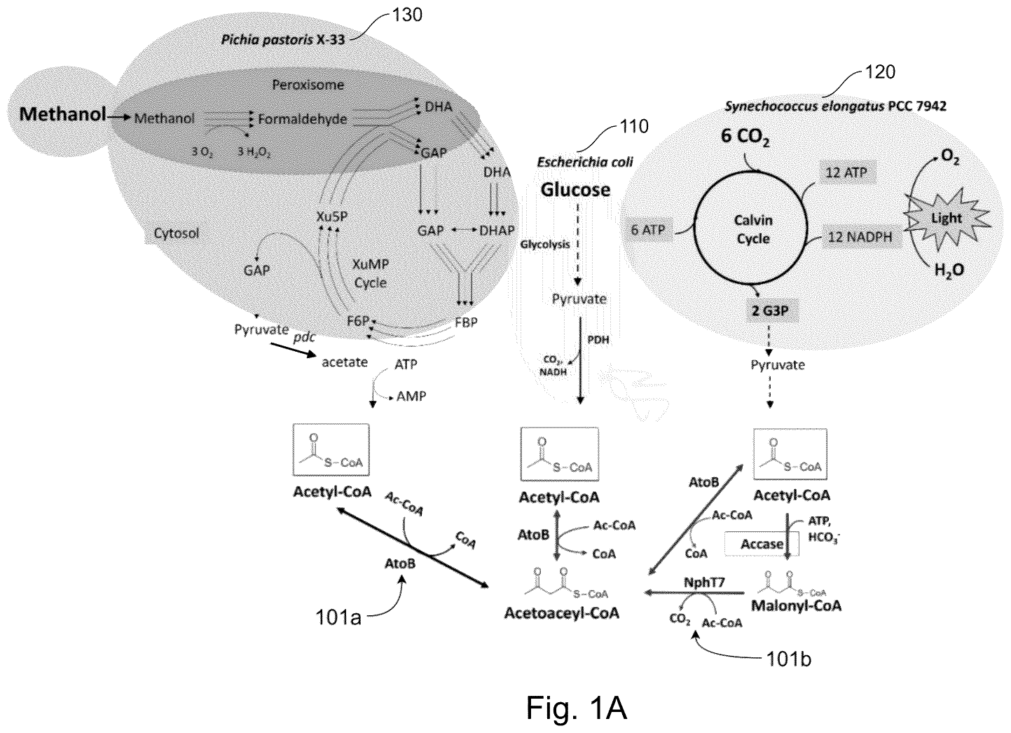

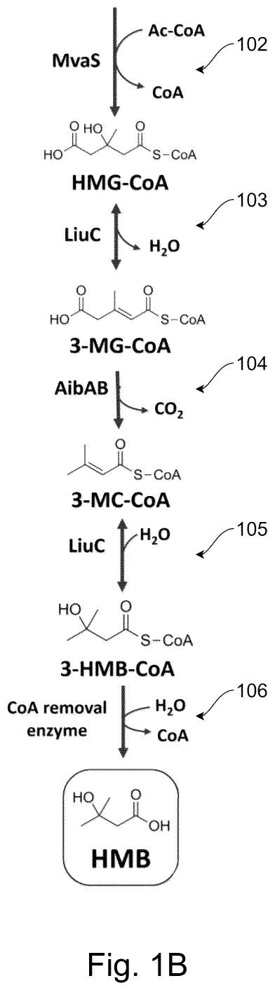

In this embodiment, the microbes to be used include Escherichia coli, Synechococcus elongatus, and Pichia pastoris. FIGS. 1A-1B show the designed synthetic metabolic pathway of 3-hydroxy-3-methylbutyrate within microorganisms.

In FIG. 1A, three different types of microorganisms are shown: Escherichia coli 110, Synechococcus elongatus (strain PCC 7942) 120, and Pichia pastoris (strain X-33) 130. Each microorganism utilizes different compounds as carbon sources. They first synthesize acetyl-CoA within their respective metabolic pathways and then obtain acetoacetyl-CoA through the action of acetyl-CoA acetyltransferase in reaction step 101a. Escherichia coli 110, a prokaryotic microorganism, utilizes glucose as its carbon source, obtaining acetyl-CoA through glycolysis. Pichia pastoris 130, a higher eukaryotic organism, utilizes methanol as its carbon source, and after a series of synthetic metabolic steps, sequentially produces pyruvate, acetate, and acetyl-CoA.

Synechococcus elongatus 120 is a photosynthetic microorganism capable of utilizing carbon dioxide directly. Through the Calvin cycle of photosynthesis, Synechococcus elongatus 120 first produces pyruvate, and then, via a series of synthetic metabolic steps, obtains acetyl-CoA. Synechococcus elongatus 120 serves as a model organism with abundant genetic tools. Hence in reaction step 101b, it is designed to synthesize acetoacetyl-CoA from acetyl-CoA, via the expression of acetyl-CoA thiolase encoded by the nphT7 gene from the Streptomyces species. In FIG. 1B, a series of synthetic metabolic pathways, including reaction steps 102-106, are designed from acetoacetyl-CoA to 3-hydroxy-3-methylbutyric acid.

Since Escherichia coli 110, Synechococcus elongatus 120, and Pichia pastoris 130 do not naturally possess all the enzymes required for the synthesis pathway of 3-hydroxy-3-methylbutyric acid (HMB) as depicted in FIG. 1A-1B, gene transfer must be performed on these microorganisms. Therefore, if any of the enzymes required in the HMB synthesis pathway in FIG. 1A-1B are missing or expressed insufficiently in Escherichia coli 110, Synechococcus elongatus 120, or Pichia pastoris 130, plasmids are designed to carry the genes encoding these deficient or insufficiently expressed enzymes for gene transfer into these microorganisms.

Table 1 outlines the enzymes required for the synthesis metabolic pathway of 3-hydroxy-3-methylbutyric acid (HMB) as depicted in FIGS. 1A-1B, along with the genes from the respective microbial sources Escherichia coli 110, Synechococcus elongatus 120, and Pichia pastoris 130 that need gene transfer. It also lists the substrates and products of each enzyme, along with their abbreviations as shown in FIGS. 1A-1B. The details of the gene transfer for Synechococcus elongatus 120, and Pichia pastoris 130 will be elaborated in subsequent examples. Table 2 presents the amino acid sequences of the enzymes listed in Table 1.

TABLE 1
Enzymes requiring gene transfer for the synthesis metabolic pathway of 3-hydroxy-
3-methylbutyric acid as depicted in FIGS. 1A-1B, along with other relevant information
for Escherichia coli, Synechococcus elongatus, or Pichia pastoris.

| Source | | | |
|---|---|---|---|
| Microorganism | Enzyme | Substrate | Product |
| E. coli | AtoB: | Acetyl-CoA | Acetoacetyl-CoA |
| | Acetyl-CoA | (Ac-CoA) | (AcAc-CoA) |
| | acetyltransferase | | |
| Streptomyces sp. | NphT7: | Malonyl-CoA | Acetoacetyl-CoA |
| | Acetoacetyl-CoA thiolase | | (AcAc-CoA) |
| Staphylococcus | MvaS: | Acetoacetyl-CoA | 3-hydroxy-3- |
| aureus | Hydroxymethylglutaryl- | (AcAc-CoA) | methylglutaryl- |
| | CoA synthase | | CoA |
| | | | (3-HMG-CoA) |
| Stigmatella | LiuC: | 3-hydroxy-3- | 3- |
| aurantiaca | 3-hydroxy-3- | methylglutaryl- | methylglutaconyl- |
| | methylglutaconyl-CoA | CoA | CoA |
| | dehydratase | (3-HMG-CoA) | (3-MG-CoA) |
| | AibAB: Composed of | 3- | 3-methyl crotonyl- |
| | AibA and AibB | methylglutaconyl- | CoA |
| | 3-methylglutaconyl-CoA | CoA | (3-MC-CoA) |
| | decarboxylase | (3-MG-CoA) | |
| | LiuC: | 3-methyl crotonyl- | 3-hydroxy-3- |
| | 3-hydroxy-3- | CoA | methylbutyryl-CoA |
| | methylglutaconyl-CoA | (3-MC-CoA) | (3-HMB-CoA) |
| | dehydratase | | |
| E. coli | YciA, TesB, MenI [symbol] | 3-hydroxy-3- | 3-hydroxy-3- |
| | YqiA: | methylbutyryl-CoA | methylbutyric acid |
| | thioester hydrolase | (3-HMB-CoA) | (HMB) |
| | or thioesterase | | |

TABLE 2
The amino acid sequences of the enzymes listed in Table 1.

| SEQ | | |
|---|---|---|
| ID | | |
| NO: | Enzyme | Amino acid sequence |
| 1 | AtoB | MKNCVIVSAV RTAIGSFNGS LASTSAIDLG ATVIKAAIER |
| | | AKIDSQHVDE VIMGNVLQAG LGQNPARQAL LKSGLAETVC |
| | | GFTVNKVCGS GLKSVALAAQ AIQAGQAQSI VAGGMENMSL |
| | | APYLLDAKAR SGYRLGDGQV YDVILRDGLM CATHGYHMGI |
| | | TAENVAKEYG ITREMQDELA LHSQRKAAAA IESGAFTAEI |
| | | VPVNVVTRKK TFVFSQDEFP KANSTAEALG ALRPAFDKAG |
| | | TVTAGNASGI NDGAAALVIM EESAALAAGL TPLARIKSYA |
| | | SGGVPPALMG MGPVPATQKA LQLAGLQLAD IDLIEANEAF |
| | | AAQFLAVGKN LGFDSEKVNV NGGAIALGHP IGASGARILV |
| | | TLLHAMQARD KTLGLATLCI GGGQGIAMVI ERLN |
| 2 | NphT7 | MTDVRFRIIG TGAYVPERIV SNDEVGAPAG VDDDWITRKT |
| | | GIRQRRWAAD DQATSDLATA AGRAALKAAG ITPEQLTVIA |
| | | VATSTPDRPQ PPTAAYVQHH LGATGTAAFD VNAVCSGTVF |
| | | ALSSVAGTLV YRGGYALVIG ADLYSRILNP ADRKTVVLFG |
| | | DGAGAMVLGP TSTGTGPIVR RVALHTFGGL TDLIRVPAGG |
| | | SRQPLDTDGL DAGLQYFAMD GREVRRFVTE HLPQLIKGFL |
| | | HEAGVDAADI SHFVPHQANG VMLDEVFGEL HLPRATMHRT |
| | | VETYGNTGAA SIPITMDAAV |
| | | RAGSFRPGEL VLLAGFGGGM AASFALIEW |
| 3 | MvaS | MTIGIDKINF YVPKYYVDMA KLAEARQVDP NKFLIGIGQT |
| | | EMAVSPVNQD IVSMGANAAK DIITDEDKKK IGMVIVATES |
| | | AVDAAKAAAV QIHNLLGIQP FARCFEMKEA CYAATPAIQL |
| | | AKDYLATRPN EKVLVIATDT ARYGLNSGGE PTQGAGAVAM |
| | | VIAHNPSILA LNEDAVAYTE DVYDFWRPTG HKYPLVDGAL |
| | | SKDAYIRSFQ QSWNEYAKRQ GKSLADFASL CFHVPFTKMG |
| | | KKALESIIDN ADETTQERLR SGYEDAVDYN RYVGNIYTGS |
| | | LYLSLISLLE NRDLQAGETI GLFSYGSGSV GEFYSATLVE |
| | | GYKDHLDQAA HKALLNNRTE VSVDAYETFF KRFDDVEFDE |
| | | EQDA VHEDRH IFYLSNIENN VREYHRPE |
| 4 | LiuC | MPEFKVDARG AIEIWTIDGA DRRNAISRAM LQELSGMVTR |
| | | VSTGRAVRAV IITGAGDKAF CAGADLKERA GMSEAEVRAF |
| | | LDGLRQTLRA IEKSDCVFIA AINGAALGGG TELSLACDLR |
| | | VAVPATELGL TEVRLGIIPG GGGTQRLSRL VGPGRAKDLI |
| | | LTGRRINAAE AFSIGLVNRL APEGHLVETS FSLAEAIVAN |
| | | APIAVSTAKH AIDEGTGLEL DDALALELRK YEDILQTEDR |
| | | LEGLRSFAEK RPPVYKGR |
| 5 | AibA | MRFHGWRPLS EAVASISDGA WLATGGFMLG RAPMALVLEL |
| | | IAQKROGLRL ISLPNPLPAE FLVAGGCLAH VDLPFGALNL |
| | | EGRVRPMPCL KRAIEQNRLS WREHDGYRVV QRLRAASMGL |
| | | PFLPAPDTDV SALASAEPPR TVVDPFTGQT VTVEPAFYPD |
| | | VALVHAQAAD ERGNLYIEDP TTDLLVAGAA RRVIATAEQR |
| | | VPRLERVTVP GFQVESVSLA PRGALPTGCL GLYAHDDAML |
| | | AHYLELAEAG REAEFLSRLL DARRAA |
| 6 | AibB | MSTPSEATPA ETVVALLARE IEDGAVIATG VASPLAILAI |
| | | AVARATHAPR LTYLACVGSL DPALPTLLPS SEDLGYLLGR |
| | | TAEITIADLF DHARRGRVDT IFFGAAEVDA RGRTNMTAAG |
| | | SLERPRVKFP GVAGAATLRQ WVRRPVLLVP KQSRRNLVPE |
| | | VQVATTQDPR RPVRLISDLG IFELGAEGAR LHARHAWATA |
| | | ADISERTGFS FSVAAPLPVT PPPDARTLEA IRTIDSHCFR DQLVGA |
| 7 | YciA | MSTTHNVPQG DLVLRTLAMP ADTNANGDIF GGWLMSQMDI |
| | | GGAILAKEIA HGRVVTVRVE GMTFLRPVAV GDVVCCYARC |
| | | VQKGTTSVSI NIEVWVKKVA SEPIGQRYKA TEALFKYVAV |
| | | DPEGKPRALP VE |
| 8 | TesB | MSQALKNLLT LLNLEKIEEG LFRGQSEDLG LRQVFGGQVV |
| | | GQALYAAKET VPEERLVHSF HSYFLRPGDS KKPIIYDVET |
| | | LRDGNSFSAR RVAAIQNGKP IFYMTASFQA PEAGFEHQKT |
| | | MPSAPAPDGL PSETQIAQSL AHLLPPVLKD KFICDRPLEV |
| | | RPVEFHNPLK GHVAEPHRQV WIRANGSVPD DLRVHQYLLG |
| | | YASDLNFLPV ALQPHGIGFL EPGIQIATID HSMWFHRPFN |
| | | LNEWLLYSVE STSASSARGF VRGEFYTQDG VLVASTVQEG |
| | | VMRNHN |
| 9 | MenI | MIWKRKITLE ALNAMGEGNM VGFLDIRFEH IGDDTLEATM |
| | | PVDSRTKQPF GLLHGGASVV LAESIGSVAG YLCTEGEQKV |
| | | VGLEINANHV RSAREGRVRG VCKPLHLGSR HQVWQIEIFD |
| | | EKGRLCCSSR LTTAIL |
| 10 | YqiA | MSTLLYLHGF NSSPRSAKAS LLKNWLAEHH PDVEMIIPQL |
| | | PPYPSDAAEL LESIVLEHGG DSLGIVGSSL GGYYATWLSQ |
| | | CFMLPAVVVN PAVRPFELLT DYLGQNENPY TGQQYVLESR |
| | | HIYDLKVMQI DPLEAPDLIW LLQQTGDEVL DYRQAVAYYA |
| | | SCRQTVIEGG NHAFTGFEDY FNPIVDFLGL HHL |

Experiment 1: Cultivation of Escherichia coli

Unless otherwise stated, all chemicals were purchased from TCI (Portland, OR). J. T. Baker (Phillipsburg. NJ. USA), or Sigma-Aldrich (St. Louis, MO. USA). Complex media such as Luria broth (LB), yeast extract, and LB agar were obtained from FocusBio (Guangzhou, China). All seed cultures were grown in LB medium at 37° C. with shaking at 200 rpm in a shaking incubator.

The culture medium used for HMB-producing strains was M9 minimal medium supplemented with glucose at a concentration below 40 g/L. The composition of M9 medium per liter included 12.8 g of Na₂HPO₄·7H₂O, 3 g of KH₂PO₄, 0.5 g of NaCl, 0.5 g of NH₄Cl, 1 mM of MgSO₄, 1 mg/mL of vitamin B1, and 0.1 mM of CaCl₂).

Additionally, when necessary, an appropriate concentration of antibiotics was added to the medium to facilitate strain selection. For example, 50 mg/mL kanamycin (kan), 100 μg/mL ampicillin (Amp), or 50 μg/mL chloramphenicol (Cm) could be added.

For strains with the glta gene knocked out, the medium used contained less than 40 g/L glucose and an additional nitrogen source compound at 5 g/L.

The cell growth of the strains was measured at a wavelength of 600 nm (OD₆₀₀) using a Biotek Epoch 2 microplate spectrophotometer, with the optical path length adjusted to 1.

Experiment 1-2: Gene Electroporation in Escherichia coli

The entire process of electroporation was basically conducted on ice.

The 1% (V/V) bacterial suspension of E. coli cultured overnight was inoculated into a shaking flask containing 20 mL of LB medium. When the bacterial suspension in the flask reached an OD₆₀₀ of 0.4 to 0.6, it was placed on ice and transferred to a 50 mL Falcon tube, then was centrifuged at 4° C. at a speed of 4,020 rpm for 10 minutes. The supernatant was discarded, and the cells were resuspended in 1 mL of 10 vol % glycerol and transferred to 2 mL microcentrifuge tubes. The tubes were centrifuged at 5,000×g for 2 minutes, and this washing step was repeated three times. Finally, the cells were resuspended and concentrated 30-fold in 10 vol % glycerol.

Next, 50 μL of concentrated bacterial culture and 50-100 ng of plasmid were mixed in a 1.5 mL centrifuge tube, and then the mixture was placed into a 1 mm electroporation cuvette (BioRad) and pulsed using the Ec1 program (V=1.8 kV) on a MicroPulser (Bio-Rad), with a time constant of 5.4 to 5.9 ms. After electroporation, the cells were mixed with 200 μL of LB medium and cultured at 37° C. and 200 rpm shaking for 1 hour. Subsequently, 100 μL and 1 μL of the bacterial solution were plated on LB agar plates containing the corresponding antibiotics.

The next day, three individual colonies were randomly picked from the LB agar plates to serve as three biological replicates, and each was inoculated into 1 mL of LB broth. The overnight cultures were then inoculated at 1% (V/V) into 2 mL of M9 medium containing glucose at a concentration below 40 g/L and the corresponding antibiotics.

Experiment 1-3: Fermentation of Escherichia coli in a Bioreactor

The fermentation medium for E. coli utilized M9 medium supplemented with trace metals and 40 g/L of glucose. The salt composition per liter of M9 medium included 6 g of Na₂HPO₄, 3 g of KH₂PO₄, 0.5 g of NaCl, and 1 g of NH₄Cl. Additionally, the M9 medium contained 1 mM of MgSO₄, 0.1 mg/mL of thiamine, 0.1 mM of CaCl₂), 40 g/L of glucose, and 10 ml/L of trace metal solution.

The trace metal solution was prepared by dissolving 5 g of EDTA in 800 mL of water and adjusting the pH to 7.5 using 3M NaOH. Subsequently, the remaining trace metals were added, and water was added to achieve a final volume of 1 liter. The concentrations of the remaining trace metals were as follows: 498 mg FeCl₃ and 84 mg ZnCl₂ per liter of water, along with 0.1 M Cu Cl₂·2H₂O, 0·2 M Co Cl₂·6 H₂O, 0.1 M H₃BO₃, and 1 M MnCl₂·4H₂O per liter of trace metal solution.

Two days before fermentation, seed cultures were prepared following the steps below. Firstly, a single colony was randomly selected from an LB plate and inoculated into test tubes containing 4 mL of LB broth. These tubes were then shaken at 37° C. overnight. The following day, 2.5% (V/V) of the overnight culture was inoculated into shake flasks containing 50 mL of fermentation medium, and the flasks were shaken at 37° C. for 12-14 hours.

The fermentation was conducted in a 5 L bioreactor (Biostat® A, Sartorius) using a fed-batch strategy. The working volume of the bioreactor was 1 L. After centrifugation of the seed culture, the seed culture was concentrated using the fermentation medium. The fermentation started with an initial OD₆₀₀ of 0·2. The pH was automatically controlled by a computer-controlled pump with 3 M NaOH to maintain a pH of 7.0, and the dissolved oxygen level was set to 30%. When the OD₆₀₀ reached 2, 0.1 mM Isopropyl β-D-1-thiogalactopyranoside (IPTG) was added to induce the expression of RNA polymerase in E. coli for the expression of exogenous genes, followed by shaking the cultivation at 30° C. The feeding solution for the bioreactor consisted of M9 minimal medium containing a trace metal solution, supplemented with 50 wt % glucose, 0.1 mM IPTG, and 50 μg/mL kanamycin. Additionally, 0.02 M (NH₄) 2SO₄ was fed into the bioreactor every 12 hours as a nitrogen source for E. coli growth until the bacteria entered the stationary growth phase.

Experiment 1-4: Quantification and Analysis of Fermentation Products in Escherichia coli

1 mL of the bacterial culture was sampled into a 1.5 mL centrifuge tube and centrifuged at 18,000×g for 2 minutes. After centrifugation, the supernatant was collected for subsequent product analysis.

Non-volatile compound analysis in the fermentation products of Escherichia coli was primarily conducted using High-Performance Liquid Chromatography (HPLC). With the Shimadzu LC-2030C HPLC system, most compounds in the supernatant could be separated and purified, followed by analysis using Photodiode Array Detector (PDA) and Refractive Index Detector (RID-20A). The operating conditions included 5 mM H₂SO₄ as the mobile phase with a flow rate of 0.6 mL/min and an injection volume of 20 μL. Analysis was carried out using a Bio-Rad Aminex HPX-87H HPLC organic acid column and a Micro-Guard Cation H⁺ guard column at 35° C. Specifically, HMB, acetic acid, and glucose were detected using the RID detector, while other compounds were analyzed based on their absorbance at 210 nm using the PDA detector. Standard curves were established using 0.1-20 mM standard solutions to determine the concentrations of various products in the supernatant. The glucose consumption by E. coli was determined by the difference between the initial concentration and the final concentration of glucose in the culture medium of the experimental group.

For volatile chemical compounds such as ethanol in the fermentation products of Escherichia coli, quantification was performed using a Shimadzu GC-2010 Gas Chromatograph (GC) equipped with a Flame Ionization Detector (FID). The gas chromatography column used was an SH-Rtx-wax column (30 m×0.32 mm, df 0.5 μm). The column oven temperature was initially held at 40° C. for 2 minutes, then ramped at a gradient of 45° C. per minute to 85° C. and held for 2 minutes, followed by a continuous increase at a rate of 45° C. per minute until reaching 230° C., and held for 1 minute. The FID detector was maintained at 230° C., while the injector temperature was set at 225° C. Nitrogen was used as the carrier gas, with a split ratio of 1:15, for injecting 1 μL of the sample into the analytical column. 1-pentanol was used as the internal standard.

Experiment 1-5: Utilization of Escherichia coli Dual-Plasmid Expression System I with Homologous Genes carried by the Second Plasmid

In this experiment, in addition to using the pRM09 plasmid carrying genes atoB, mvaS, liuC, aibA, and aibB corresponding to reaction steps 101a-105 in FIGS. 1A-1B as the first plasmid, the remaining genes capable of catalyzing reaction step 106 in FIG. 1B were selected from the Escherichia coli (E. coli K-12) in ASKA clone library and inserted into the second plasmid. The genes liuC, aibA, and aibB in the pRM09 plasmid are derived from Myxococcus xanthus, and the ampicillin resistance gene AmpR is designed in the plasmid for screening of successfully transformed strains.

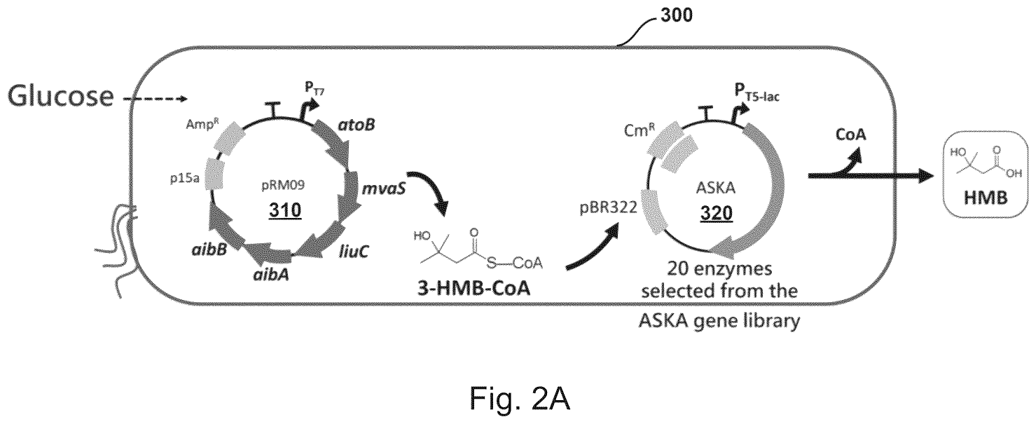

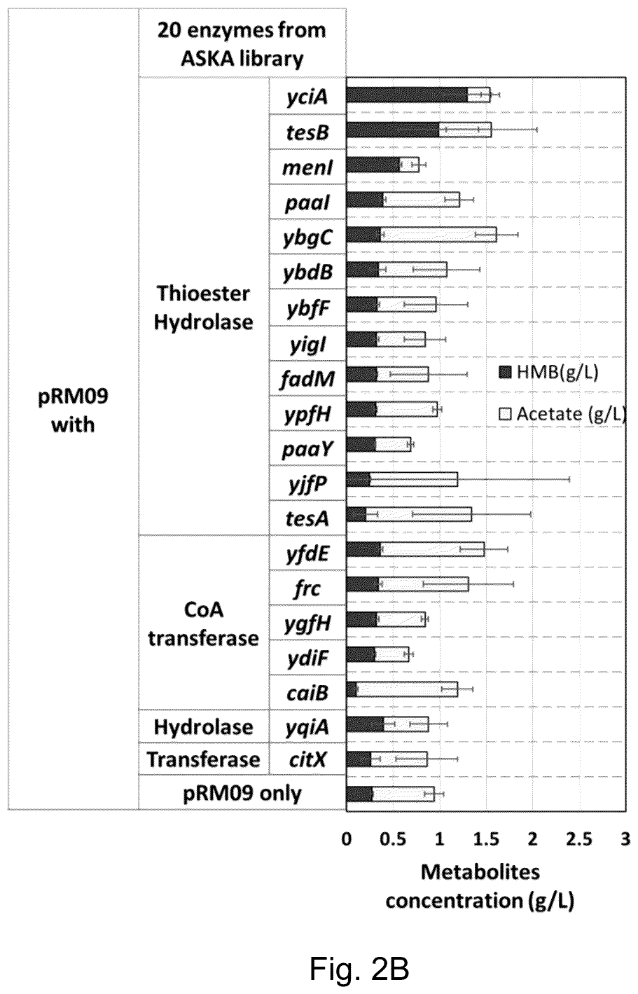

FIG. 2A illustrates a schematic diagram of establishing a dual-plasmid system for the synthetic metabolic pathway of 3-hydroxy-3-methylbutyrate in Escherichia coli. In E. coli 300 depicted in FIG. 2A, the first plasmid 310 is the pRM09 plasmid, while the second plasmid 320 carries one of the 20 enzymes screened from the ASKA clone library of E. coli for reaction step 106 in FIG. 1B. This is aimed at enhancing the reaction rate of reaction step 106 and consequently increasing the yield of the final product, HMB. Both the first plasmid 310 and the second plasmid 320 were introduced into E. coli using the gene transfer method described in Experiment 1-2.

FIG. 2B depicts the screening of various homologous genes from the ASKA clone library in Escherichia coli, capable of effectively enhancing the production of HMB in reaction step 106 of FIG. 1B. It also illustrates the concentrations of HMB and acetate in the bacterial solution after 24 hours of fermentation. In FIG. 2B, it can be observed that among the genes expressed by E. coli after inserting various homologous enzymes into the second plasmid 320, those encoding thioesterases such as yciA, tesB, menI, and yqiA, as well as the hydrolyase encoded by the yqiA gene, exhibit higher catalytic efficiency for reaction step 106 of FIG. 1B, resulting in higher concentrations of HMB. After 24 hours of fermentation, the concentrations of HMB obtained from the expression of thioesterases encoded by yciA, tesB, menI, and yqiA genes, as well as the hydrolyase encoded by the yqiA gene, were 1·29, 0.99, 0.56, and 0.4 g/L, respectively.

### Example 1-6: Utilization of the Escherichia coli Dual-Plasmid Expression System II to Carry Homologous Genes from Other Microorganisms on the Second Plasmid

In this experiment, the first plasmid carried genes atoB, mvaS, liuC, aibA, and aibB for the HMB synthesis pathway under the control of the PLlacO1 promoter. The genes liuC, aibA, and aibB were sourced from Myxococcus xanthus. The second plasmid was utilized to carry genes encoding thioesterases from microorganisms other than E. coli to assess their catalytic efficiency in reaction step 106 of the pathway depicted in FIG. 1B. Similarly, the HMB production yield after 24 hours of fermentation was used as the comparative analysis metric.

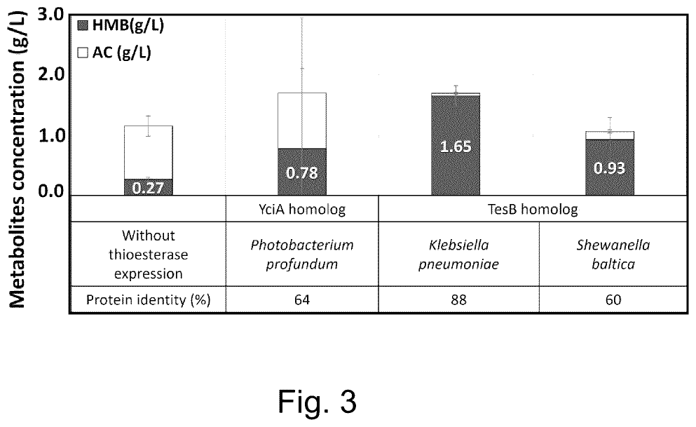

FIG. 3 illustrates a schematic diagram of the Escherichia coli dual-plasmid expression system utilizing the second plasmid to carry other microbial homologous genes, along with the concentrations of metabolites after 24 hours of fermentation. In FIG. 3, it can be observed that the homologous proteins of the thioesterases expressed from the homologous genes of yciA and tesB of other microorganisms, including Photobacterium profundum, Klebsiella pneumoniae, and Shewanella baltica, all exhibit catalytic activity in reaction step 106 of the pathway, leading to an increased production of HMB. Among them, the acyl-CoA thioesterase II from Klebsiella pneumoniae showed the optimal performance, yielding up to 1.65 g/L of HMB, with the least amount of acetic acid (AC) produced as a byproduct.

Experiment 1-7: Escherichia coli Monoplasmid Expression System

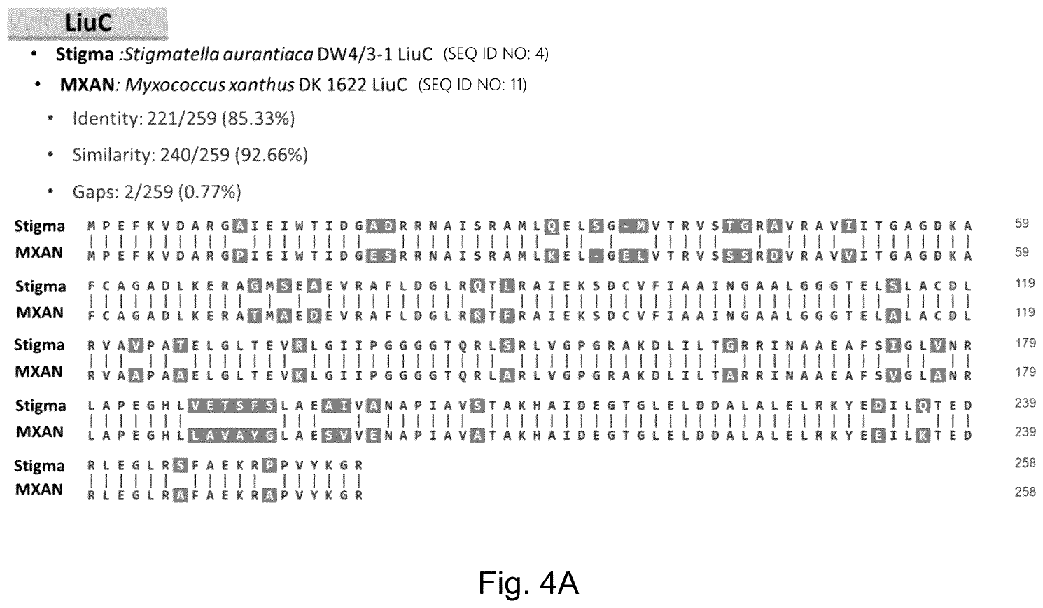

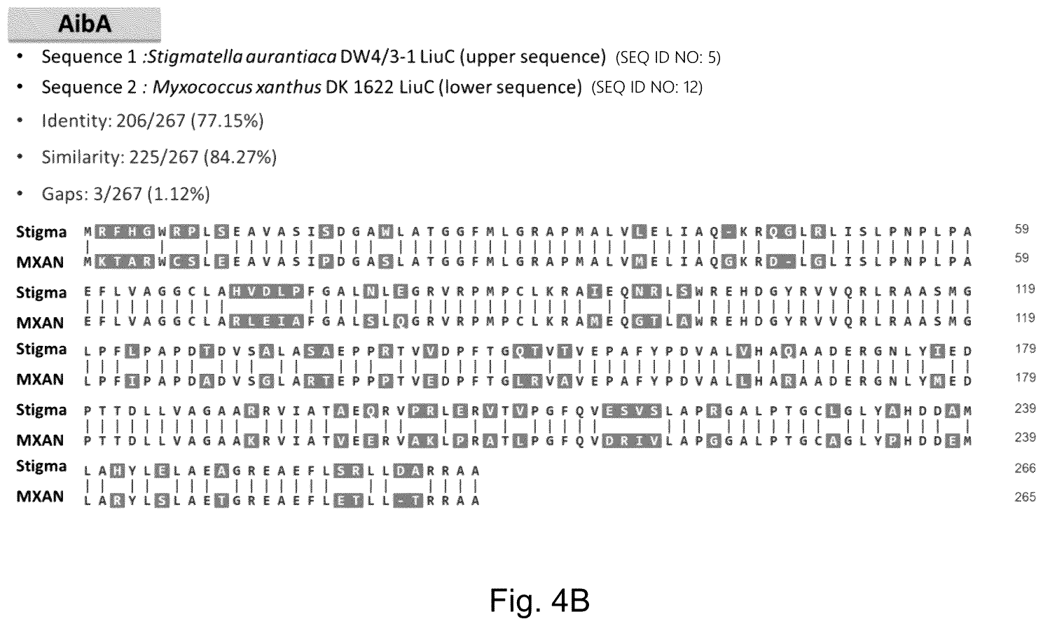

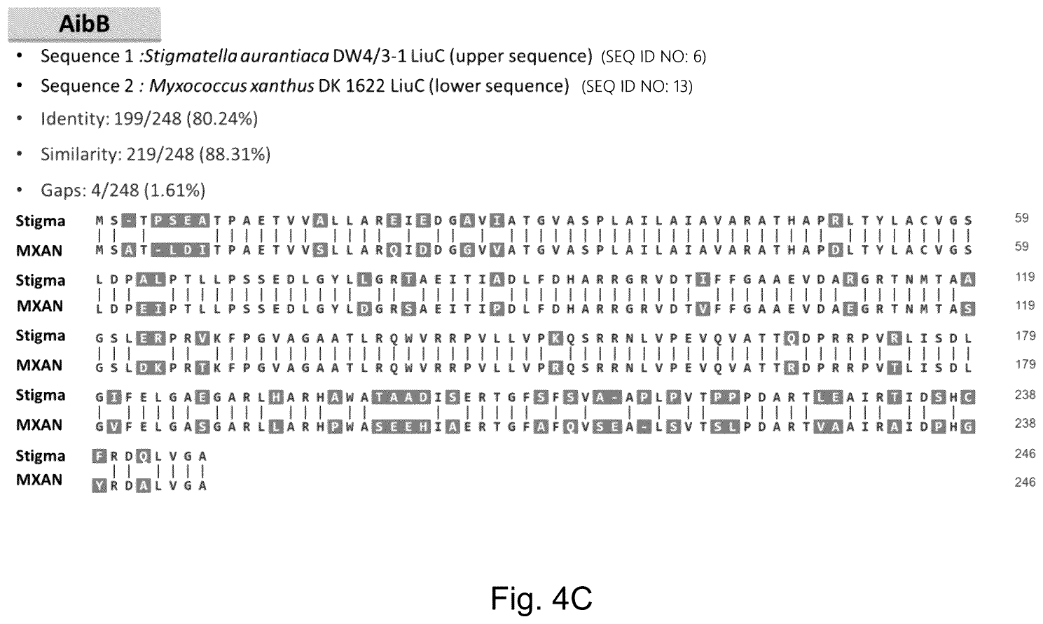

In this experiment, the PLlacO1 promoter was used to carry all genes involved in the HMB synthesis pathway, and genes responsible for reaction steps 103-105 in FIG. 1B are derived from the same homologous enzymes listed in Table 3, originating from various species within the order Myxococcales. From Table 3, it can be observed that compared to the enzymes of Stigmatella aurantiaca DW4/3-1, the homologous enzymes from other microorganisms within the order Myxococcales in Table 3 exhibit a similarity of over 50%. Additionally, FIGS. 4A-4C respectively depict the comparison of protein sites of LiuC, AibA, and AibB from strains Stigmatella aurantiaca DW4/3-1 (SEQ ID NO: 4-6) and Myxococcus xanthus DK 1622 (SEQ ID NO: 11-13), as used in Chinese patent CN107075536A.

TABLE 3
Relative to the enzymes LiuC and AibAB from the strain Stigmatella aurantiaca
DW4/3-1, the comparison of the protein identity and similarity of the
homologous enzymes LiuC and AibAB from the order Myxococcales.

| | | protein identity/similarity (%) | | |
|---|---|---|---|---|
| Order | Strain | LiuC | AibA | AibB |
| Myxococcales | Stigmatella aurantiaca DW4/3-1 | 100 | 100 | 100 |
| | Myxococcus xanthus | 85/92 | 77/84 | 80/88 |
| | Archangium gephyra DSM 2261 | 82/90 | 82/88 | 80/86 |
| | Melittangium boletus DSM 14713 | 83/89 | 81/89 | 81/88 |
| | Cystobacter fuscus DSM 2262 | 83/89 | 77/88 | 79/88 |
| | Pyxidicoccus parkwaysis | 85/91 | 80/87 | 80/87 |
| | Hyalangium minutum DSM 14724 | 88/94 | 86/92 | 79/84 |
| | Labilithrix luteola DSM 27648 | 49/65 | 68/79 | 68/81 |

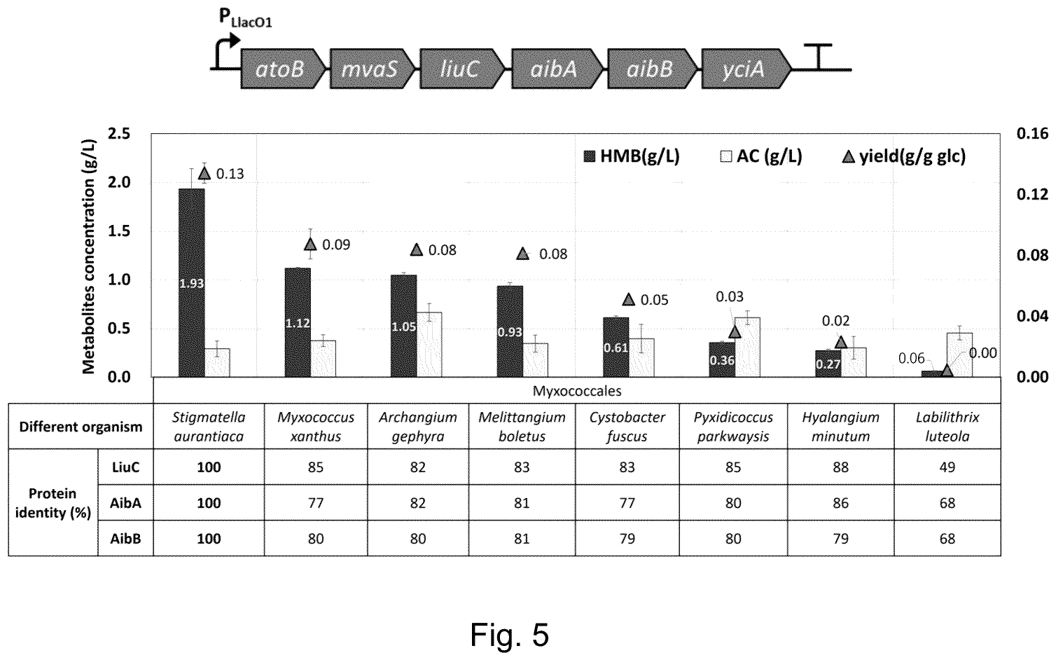

FIG. 5 illustrates the establishment of a single-plasmid system in Escherichia coli for expressing the HMB synthesis pathway and replacing the species sources of the genes liuC, aibA, and aibB. It compares the production and yield of HMB and the main byproduct acetate (AC) after 24 hours of fermentation in various strains. In FIG. 5, it can be observed that upon carrying the homologous enzymes of the genes liuC, aibA, and aibB of various species listed in Table 3 on a single plasmid in Escherichia coli, each species produced HMB. Among them, optimal expression was observed in Stigmatella aurantiaca DW4/3-1, which could elevate the 24-hour yield of HMB to approximately 1.93 g/L and the 24 hours productivity to around 0.13 g/g glucose. This experiment demonstrated for the first time that the homologous enzymes LiuC, AibA, and AibB from the order Myxococcales also possess the ability to produce HMB, and comparisons were made regarding the yield and productivity among various species.

Experiment 1-8: Single-plasmid Expression System III in Escherichia coli of Different Strains

In this experiment, a plasmid containing the PLlacol expression system regulated by the lacI gene, which can be expressed in most Escherichia coli strains, was constructed to carry the aforementioned genes atoB, mvaS, liuC, aibA, and aibB. The constructed P_{Llaco1} expression system plasmid was then transferred into different E. coli strains, and the HMB production of each strain was observed after 24 hours of fermentation.

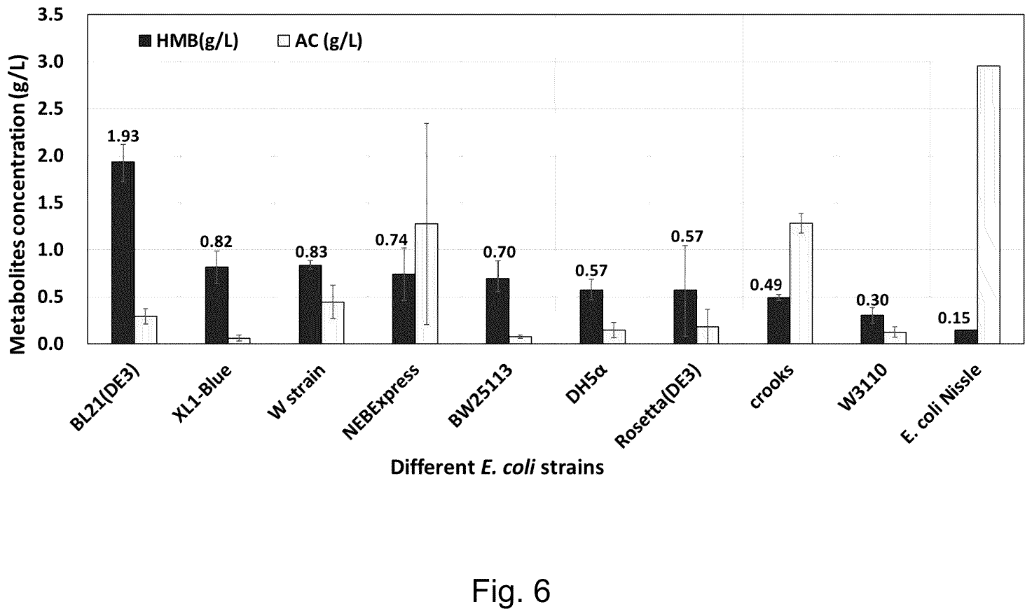

FIG. 6 depicts the production of HMB and the byproduct acetate (AC) after 24 hours of fermentation in different substrains of Escherichia coli expressing the HMB synthesis pathway. In FIG. 6, it was demonstrated that 10 different substrains of Escherichia coli were capable of producing HMB. Among them, E. coli BL21 (DE3) exhibited a HMB yield of 1.93 g/L, which was comparable to the HMB yield obtained using the PLlacO1 dual-plasmid system in Experiment 1-6 (1.65 g/L), making it the highest producer of HMB among the substrains in FIG. 6. The other substrains of E. coli included in FIG. 6 are BW25113, XL1-Blue, DH5a, W3110, NEBExpress®, Rosetta (DE3), W strain, crooks, and the probiotic E. coli Nissle 1917. Since the genetically modified probiotic E. coli Nissle 1917 is also capable of producing HMB, it could be considered for formulation with protein-rich nutrients, providing a convenient oral supplement for sarcopenia patients to promote muscle growth and address issues related to insufficient muscle mass.

Experiment 1-9: The Impact of Different Culture Media on HMB Production in Escherichia coli

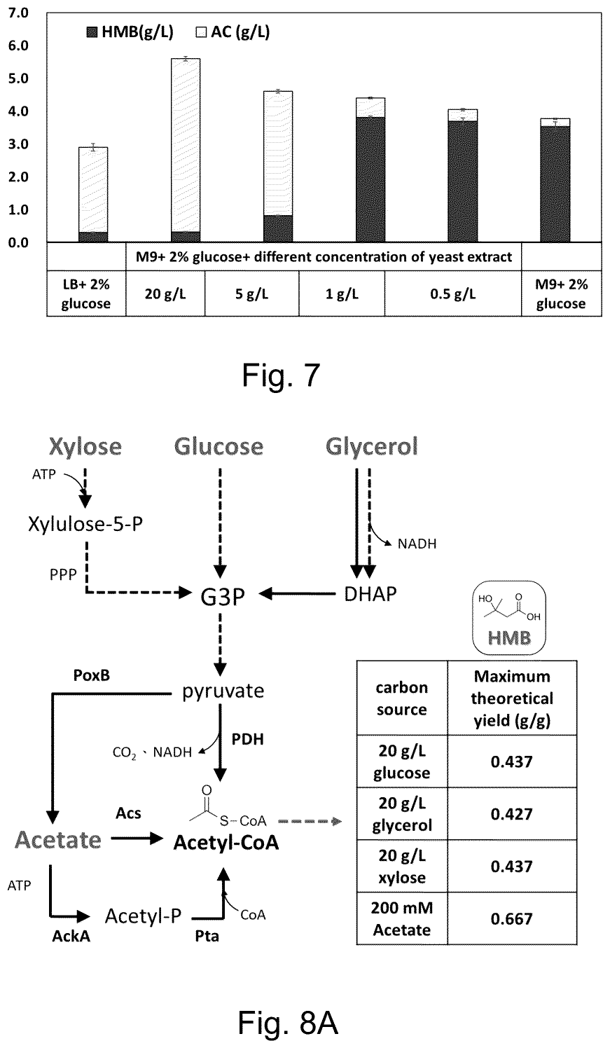

In this experiment, different culture media were used to cultivate Escherichia coli to observe the impact of different culture media on HMB production. The results obtained are shown in FIG. 7.

FIG. 7 illustrates the production of HMB and the byproduct acetate (AC) after 24 hours of fermentation in Escherichia coli cultured using complex media or synthetic media. In FIG. 7, three types of culture media were used: LB medium, M9 minimal medium supplemented with yeast extract at different concentrations (20, 5, 1, 0.5 g/L) as additional nitrogen sources, and M9 minimal medium without additional nitrogen source. All three media contained glucose at concentrations below 40 g/L as the carbon source. LB medium and M9 minimal medium supplemented with yeast extract were complex media, while M9 minimal medium without additional nitrogen source was a synthetic medium.

The results revealed that the culture medium used for HMB biosynthesis does not require the addition of any additional nitrogen sources, such as lysogeny broth (LB) or yeast extract. Instead, the highest HMB yield (approximately 3.53 g/L) and the least amount of acetic acid byproduct were achieved using the M9 minimal medium composed mainly of salts. Therefore, using only M9 minimal medium can reduce the cultivation cost of Escherichia coli and offers significant commercial advantages.

Experiment 1-10: The Influence of Different Carbon Sources on HMB Production in Escherichia coli

In this experiment, various compounds were employed as carbon sources for Escherichia coli to observe the impact of different carbon sources on HMB production.

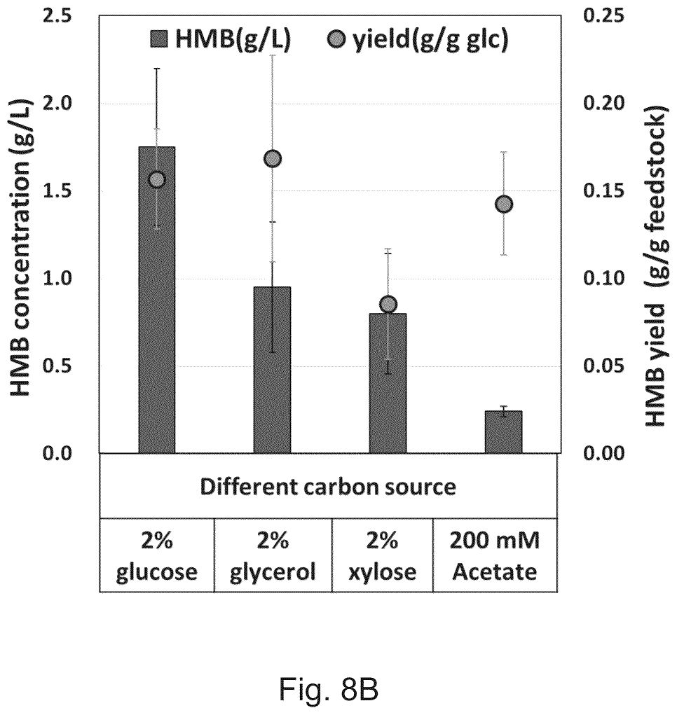

FIG. 8A depicts a schematic diagram of culturing Escherichia coli using various common carbon sources to obtain HMB through different metabolic pathways, along with the theoretical yield of HMB achievable using different carbon sources. From FIG. 8A, it was observed that apart from six-carbon glucose, Escherichia coli could also utilize five-carbon xylose and three-carbon glycerol as common carbon sources for its growth. These carbon sources were metabolized to acetyl-CoA through different metabolic pathways, which further led to the production of HMB through reaction step 101a in FIG. 1A and reaction steps 102-106 in FIG. 1B. Additionally, as HMB is a derivative of acetyl-CoA, acetate was also used as a single carbon source for E. coli in this experiment. The maximum theoretical yield of HMB from various carbon sources in FIG. 8A indicates that under conditions of adding 20 g of carbon source per liter of culture medium, the maximum theoretical yield of HMB is highest when acetate is used as the carbon source (0.667 g/g acetate), while the maximum theoretical yield of HMB is lowest when glycerol is used as the carbon source (0.427 g/g glycerol). When glucose and xylose are used as carbon sources, the maximum theoretical yield of HMB is the same, at 0.437 g/g glucose or xylose.

FIG. 8B illustrates the production and yield of HMB after 24 hours of fermentation in Escherichia coli cultured using different single carbon sources. From FIG. 8B, it was observed that culturing Escherichia coli with glucose as the sole carbon source yielded the highest HMB production, while using glycerol as the sole carbon source resulted in a higher HMB productivity. When acetate was used as the sole carbon source for culturing E. coli, there was a longer lag phase in the growth cycle, leading to lower total cell growth and HMB production. Consequently, the HMB yield obtained was the lowest, although the HMB productivity was still decent. These results indicate that other organic renewable carbon sources can be used as sole carbon sources for culturing E. coli and carrying out HMB biosynthesis.

Experiment 1-11: The Impact of Continuous Fermentation Duration on HMB Production in Escherichia coli

In this experiment, genetically modified Escherichia coli were cultivated on a large scale using a bioreactor, with a volume of 1 liter, while maintaining dissolved oxygen levels above 30%. Before adding the inducer, rapid cultivation was conducted at 37° C., followed by a temperature reduction to 30° C. after adding the inducer. Throughout the fermentation process, the pH was maintained at 7, and after a period of glucose consumption, feedings were added approximately every 50 hours to restore the glucose concentration to around 20 g/L. Under these cultivation conditions, the HMB-producing strain sustained production for approximately 11 days.

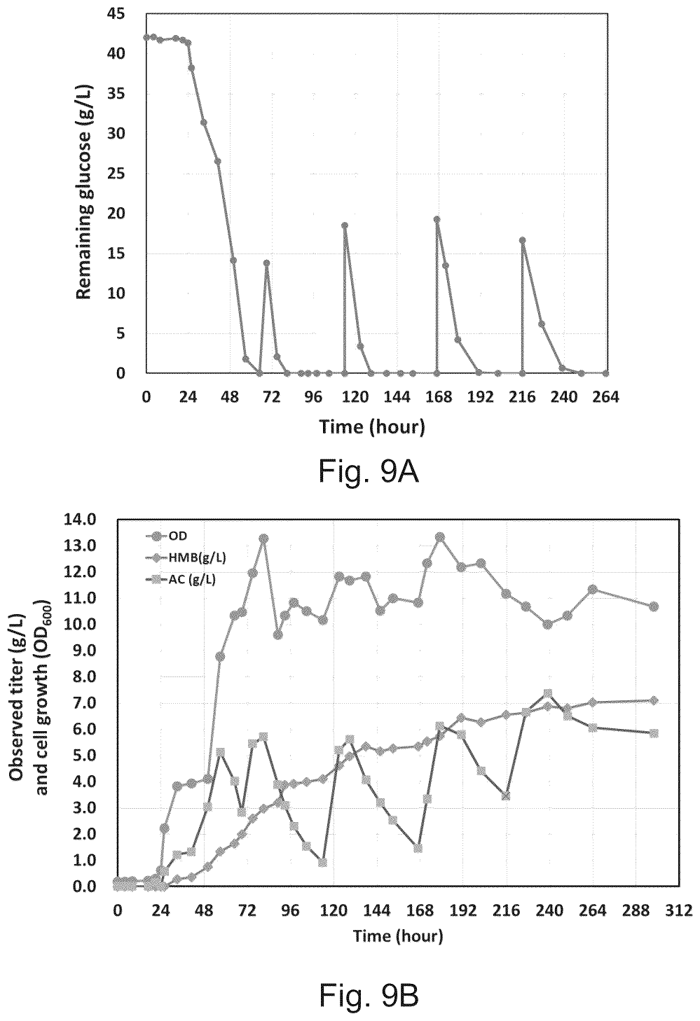

FIG. 9A depicts the time course of glucose consumption during the large-scale cultivation of transgenic Escherichia coli for HMB production in a bioreactor. From FIG. 9A, it is evident that after a period of glucose depletion, glucose was reintroduced into the bioreactor to restore the glucose concentration to approximately 20 g/L. Therefore, approximately 15-18 hours after the addition of glucose, the glucose concentration increased again to nearly 20 g/L.

FIG. 9B illustrates the time course of HMB and the main byproduct acetate (AC) production in transgenic Escherichia coli cultivated in a bioreactor for large-scale production. From FIG. 9B, it can be observed that after 11 days of fermentation of the HMB-producing strain, approximately 7.1 g/L of HMB was accumulated. Additionally, there was periodic fluctuation in the concentration of acetate. Specifically, before glucose was depleted, the acetate concentration continued to increase, and it began to gradually decrease only after glucose depletion. This indicates that acetate, as a byproduct, can serve as a carbon source for E. coli even after glucose is consumed, resulting in the continuous production of HMB. This observation is consistent with the results of Experiment 1-10, where acetate was shown to be utilized by E. coli as a carbon source.

When Escherichia coli enters a state of glucose starvation, the protein expression of RpoS increases. Acting as a regulator, the RpoS protein can modulate the transcription of hundreds of genes in Escherichia coli upon entering a state of carbon starvation, allowing the organism to resist various stresses (Peterson et al., 2005). This signifies a shift in cellular metabolism. Such a shift could potentially affect the expression of exogenous genes or the yield of products. Therefore, conventional fermentation strategies for E. coli typically aim to maintain glucose concentrations above a certain threshold. Given that producing HMB using E. coli results in significant acetate byproduct formation, this experiment was designed to allow E. coli to utilize acetate as an alternative carbon source, thereby sustaining HMB production.

Experiment 1-12: Assessing the Impact of Knocking Out Genes of Competitive Pathway on HMB Production in Escherichia coli

In this experiment, it was attempted to enhance HMB production and reduce acetate formation in Escherichia coli by knocking out genes involved in competing pathways. Both wild-type and gene-knockout strains of Escherichia coli were cultured in M9 minimal medium supplemented with less than 40 g/L glucose and 5 g/L nitrogen source compounds. The wild-type strain used in the experiment was BL21 (DE3).

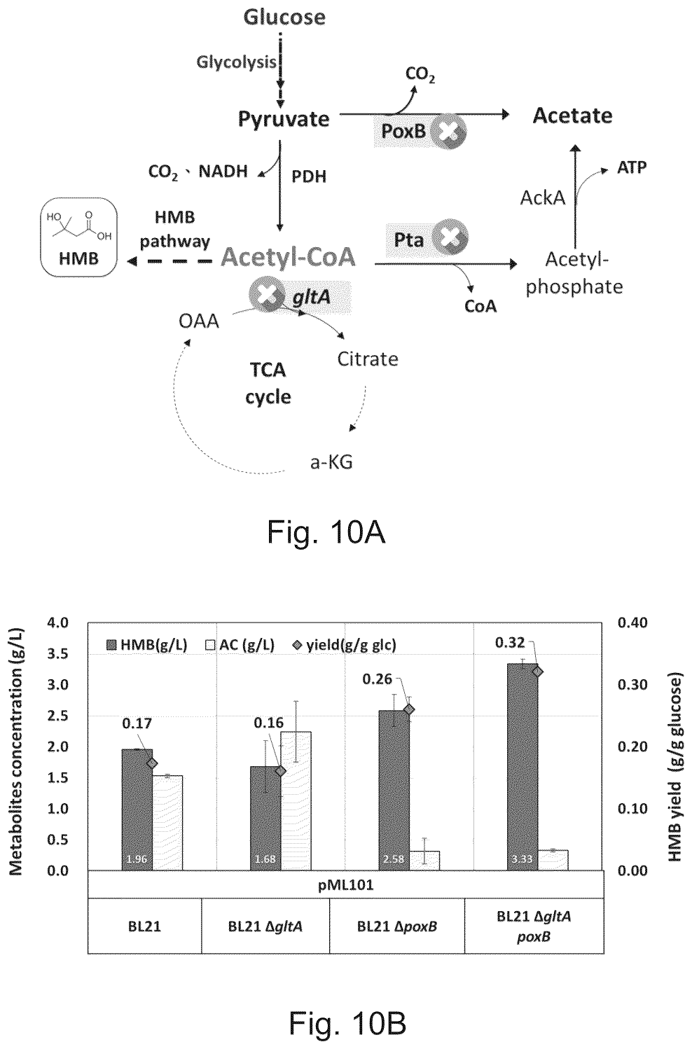

FIG. 10A illustrates a schematic diagram of competitive metabolic pathways for the production of HMB. In FIG. 10A, it is shown that acetyl-CoA is consumed by entering the TCA cycle to produce citrate, while pyruvate is metabolized to acetate. Therefore, subsequent experiments tested the knockout of competitive metabolic pathway genes, gltA (encoding citrate synthase GltA) and poxB (encoding pyruvate oxidase PoxB), or both, to observe whether it could effectively increase HMB production.

FIG. 10B shows the comparison of the 24-hour production of HMB and the main byproduct acetate (AC) in Escherichia coli producing HMB with knocking out genes from competitive pathways. From FIG. 10B, it is evident that after 24 hours of fermentation, only the E. coli strain with the knocked-out TCA cycle competitive metabolic pathway gene gltA (BL21 ΔgltA) accumulated a significant amount of acetate when expressing the HMB biosynthetic pathway. Conversely, only the E. coli strain with the knocked-out pyruvate competitive metabolic pathway gene poxB (BL21 ΔpoxB) was able to generate more HMB and less acetate.

Therefore, further testing was conducted on the expression of the E. coli strain with both genes gltA and poxB knocked out (BL21 ΔgltA ΔpoxB). The results showed that after 24 hours of fermentation, a higher HMB production was obtained. Compared to the wild-type strain, the HMB production of the E. coli strain (BL21 ΔgltA ΔpoxB) was approximately 1.7 times higher, at 3.33 g/L compared to the wild-type HMB production of 1.96 g/L. Additionally, the HMB yield of the E. coli strain (BL21 ΔgltA ΔpoxB) was approximately 1.9 times higher, at 0.32 g/g glucose compared to the wild-type HMB yield of 0.17 g/g glucose. However, the acetate production of the E. coli strain (BL21 ΔgltA ΔpoxB) was only about 15% of the wild type.

Experiment 2-1: Cultivation Method for Synechococcus elongatus PCC 7942

All Synechococcus elongatus strains were cultured using modified BG-11 medium. The composition of BG-11 medium include 1.5 g/L NaNO₃, 0.036 g/L CaCl₂)·2H₂O, 0.012 g/L ferric ammonium citrate, 0.0022 g/L Na₂EDTA·2H₂O, 0.040 g/L K₂HPO₄, 0.070 g/L MgSO₄·7H₂O, 0.020 g/L Na₂CO₃, 0.00882 g/L sodium citrate, and a 1000× dilution from a trace mineral solution (containing 1.43 g H₃BO₃, 0.905 g MnCl₂·4H₂O, 0.111 g ZnSO₄·7H₂O, 0.195 g Na₂MoO₄·2H₂O, 0.0395 g CuSO₄·5H₂O, and 0.0245 g Co(NO₃)₂·6 H₂O per 500 mL water).

Using a 1 μL inoculation loop, cells of S. elongatus cyanobacteria from a BG-11 petri dish were inoculated into fresh 40 mL modified BG-11 medium containing 50 mM NaHCO₃ and cultured in 250 mL baffled shake flasks. The cultures were then transferred to a shaking incubator (Hi-point 600SR) and grown under the following conditions: light intensity ranging from 22 to 38 μE/s/m², temperature at 30° C., and agitation at 110 rpm.

When the cell optical density at 730 nm (OD₇₃₀ nm) reached between 0.4 and 0.6, the cyanobacteria were induced to express RNA polymerase by adding IPTG to a final concentration of 1 mM, facilitating the expression of exogenous genes in the cyanobacteria. After induction, 1 mL of culture was taken out for analysis of cell growth and HMB concentration. Following sampling, 1 mL of BG-11 medium containing 500 mM NaHCO₃, appropriate antibiotics, and 1 mM IPTG was immediately added back to the culture to ensure adequate carbon supply. Cell growth was quantified by measuring absorbance at 730 nm wavelength using a Biotek Epoch 2 microplate spectrophotometer, with the path length adjusted to 1 cm.

### Experiment 2-2: Analysis of HMB Production in Genetically Modified Cyanobacteria

In this experiment, we demonstrated for the first time the feasibility of using the photoautotrophic cyanobacterium, Synechococcus elongatus PCC 7942, as a host for expressing the HMB metabolic pathway and synthesizing HMB.

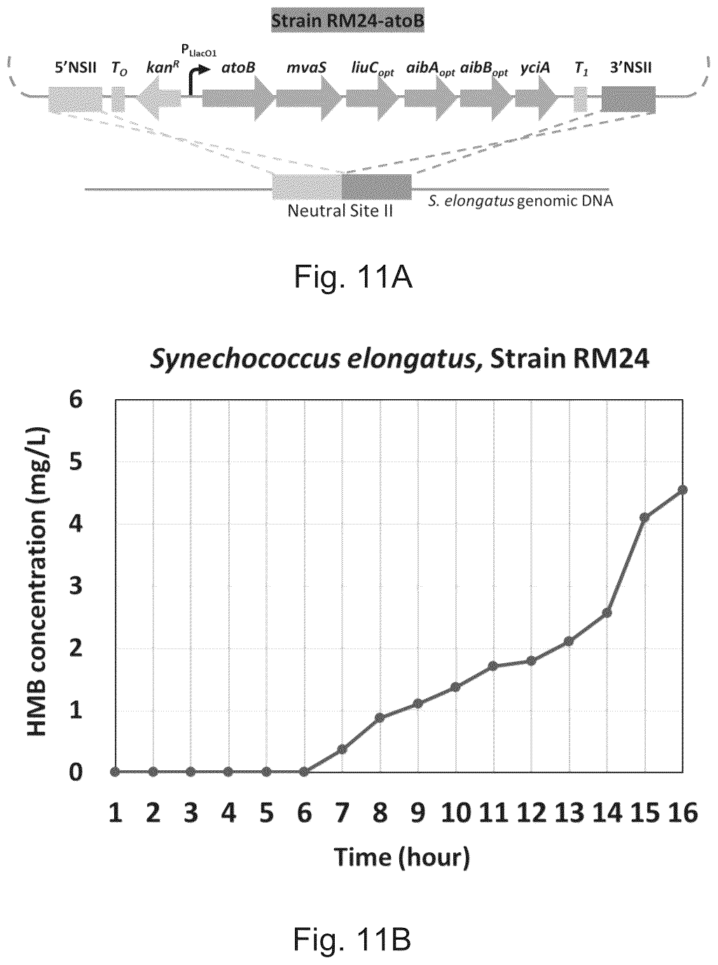

FIG. 11A illustrates a schematic diagram of the plasmid containing the complete HMB anabolic pathway genes, with NSII (Neutral site II) from the photosynthetic autotrophic cyanobacterium Synechococcus elongatus as the recombinant site. In FIG. 11A, the NSII (Neutral Site II) of Synechococcus elongatus PCC 7942 DNA was used as the recombination site, and plasmids containing the complete set of genes required for the HMB biosynthetic pathway were transferred into the RM24 strain of Synechococcus elongatus PCC 7942. After serial passage culturing, the plasmids were recombined into the genome via the recombination site. FIG. 11B depicts the variation of HMB production over time during 16 days of shake-flask cultivation of the RM24 strain of the Synechococcus elongatus PCC 7942. It can be observed from FIG. 11B that after 16 days of shake-flask cultivation, an initial HMB production of approximately 4 mg/L was achieved.

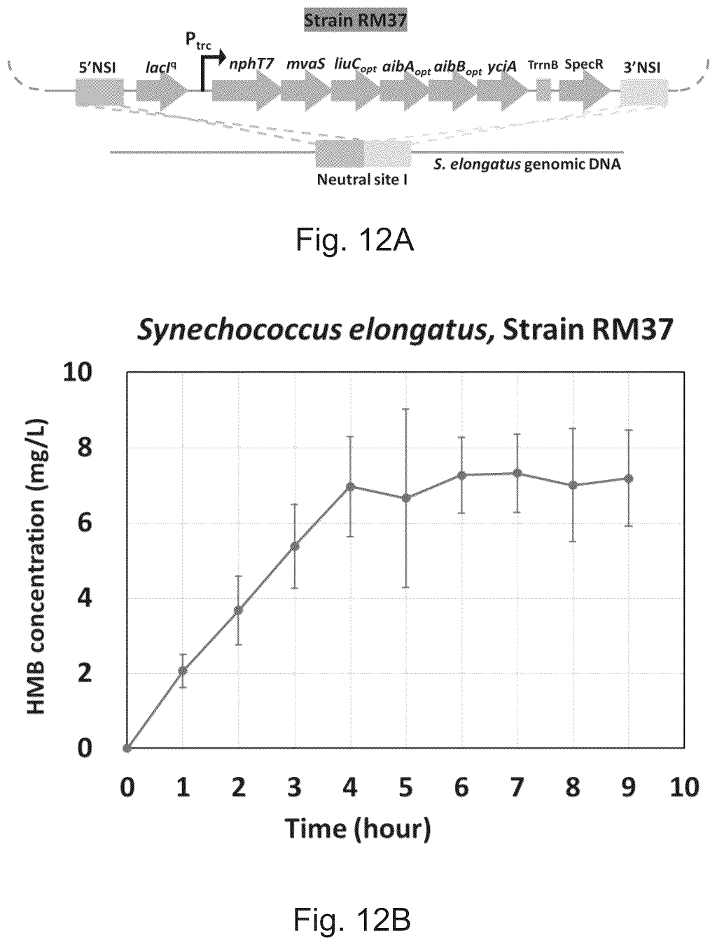

FIG. 12A illustrates a schematic diagram of the plasmid containing the complete HMB anabolic pathway genes, with NSI (Neutral site I) from the photosynthetic autotrophic cyanobacterium Synechococcus elongatus as the recombinant site. In FIG. 12A, the gene nphT7 (acetyl-CoA thioesterase) was used to replace the gene atoB (acetyl-CoA acetyltransferase) in reaction step 101a of the HMB biosynthetic pathway, resulting in the establishment of the Synechococcus elongatus PCC 7942 RM37 strain. The acetyl-CoA acetyltransferase (NphT7) catalyzes the irreversible reaction between acetoacetyl-CoA and acetyl-CoA to produce acetyl-acetyl-CoA and carbon dioxide. Strain RM37 was established in this experiment. FIG. 12B shows the time course of HMB production in the Synechococcus elongatus RM37 strain cultivated in shake flasks for 9 days. From FIG. 12B, it is evident that after 9 days of shake-flask cultivation, RM37 further achieved an HMB production of approximately 7·2 mg/L.

Experiment 3: Cultivation Method for Methylotrophic Yeast (Pichia pastoris X-33)

In this experiment, for the first time, we demonstrated the use of the eukaryotic organism, methylotrophic yeast (Pichia pastoris X-33), as a host for HMB production. Methylotrophic yeast has traditionally been widely employed as a host for heterologous protein expression systems, owing to its low production cost, capacity for post-translational modifications, and ability to regulate protein production using methanol.

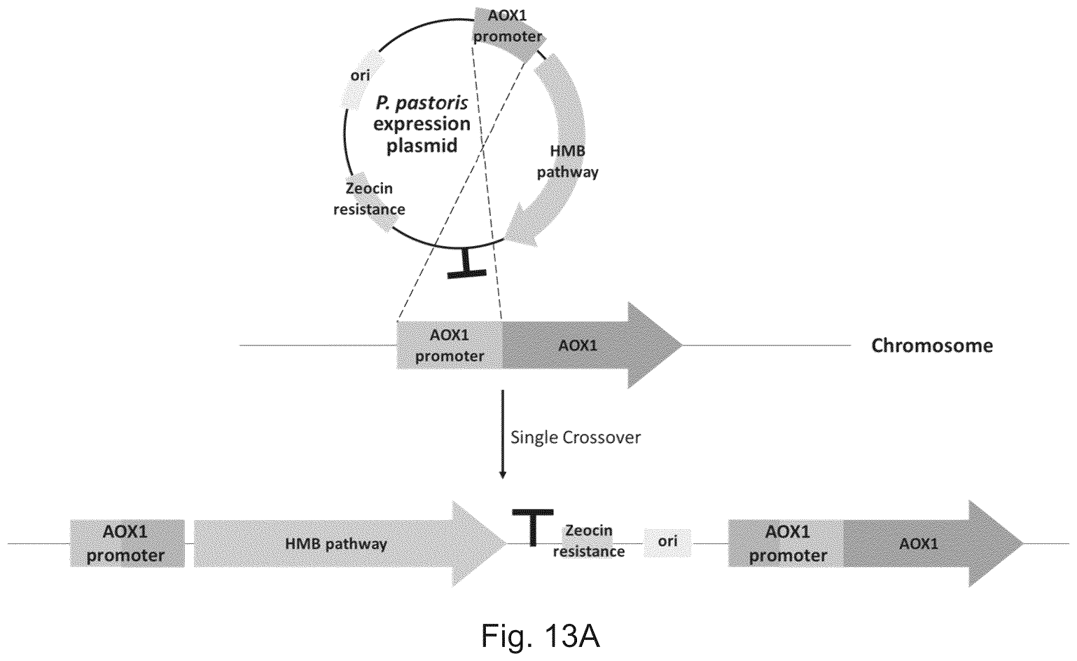

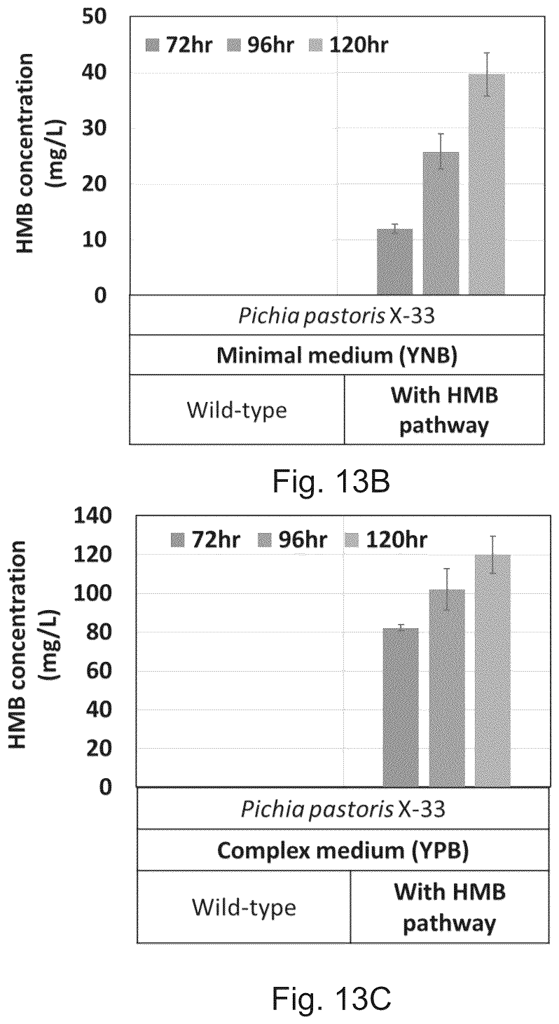

FIG. 13A illustrates a schematic diagram of the expression vector for producing HMB in the eukaryotic organism Pichia pastoris as the host. From FIG. 13A, it can be observed that the genes required for HMB biosynthesis pathway were inserted downstream of the AOX1 promoter.

Using YNB basic medium supplemented with 3 vol % methanol, the Pichia pastoris yeast was cultured in shake flasks at 30° C. and 220 rpm for 120 hours, with samples taken every 24 hours. FIG. 13B illustrates the HMB production of wild-type and genetically transformed Pichia pastoris yeast cultured in YNB basic medium for 72-120 hours. From FIG. 13B, it can be observed that after 24 hours of cultivation in YNB basic medium, the HMB production reached approximately 39.6 mg/L. The composition of the aforementioned YNB basal medium is provided in Table 4.

TABLE 4
Composition of YNB (yeast nitrogen base) basal medium

| | Ammonium sulfate: 5 g/L |
|---|---|
| Vitamins | Biotin: 2 μg/L, Calcium pantothenate: 400 μg/L, Folic acid: 2 μg/L, |
| | Inositol: 2 mg/L, Nicotinic acid: 400 μg/L, Para-aminobenzoic acid: 200 |
| | μg/L, Pyridoxine HCl: 400 μg/L, Riboflavin: 200 μg/L, Thiamine HCl: |
| | 400 μg/L |
| Trace | Boric acid: 500 μg/L, Copper sulfate: 40 μg/L, Potassium iodide: 100 |
| elements | μg/L, Ferric chloride: 200 μg/L, Manganese sulfate: 400 μg/L, Sodium |
| | molybdate: 200 μg/L, Zinc sulfate: 400 μg/L |
| Salts | Potassium dihydrogen phosphate: 1 g/L, Magnesium sulfate: 0.5 g/L, |
| | Sodium chloride: 0.1 g/L, Calcium chloride: 0.1 g/L |

In this experiment, the Pichia pastoris X-33 yeast was cultured in YPB complex medium supplemented with 3 vol % methanol at a temperature of 30° C. and a shaking speed of 220 rpm for 120 hours in shake flasks, with samples taken every 24 hours. FIG. 13C illustrated the HMB production of wild-type and genetically transformed Pichia pastoris yeast cultured in YPB complex medium for 72-120 hours. It was observed from FIG. 13C that after culturing in YPB complex medium for 120 hours, the HMB yield was approximately 119.7 mg/L. The composition of the YPB complex medium used consisted of 20 g/L peptone and 10 g/L yeast extract.

The above results indicate that using YPB complex medium for culturing Pichia pastoris yeast resulted in approximately 67% higher yield compared to using YNB basal medium. Therefore, for the production of HMB using Pichia pastoris yeast, the complex medium would be the preferable choice.

From the above embodiments of the present invention, it is evident that compared to the current chemical methods using petroleum-derived materials for preparing HMB, the embodiments of the present invention allow for microbial production of HMB directly utilizing organic carbon sources such as glucose, glycerol, and xylose. This not only reduces the dependency of the HMB market on petrochemical raw materials but also minimizes the generation of harmful by-products. Moreover, there is currently no literature or patents demonstrating that the same HMB biosynthetic pathway can be expressed in Escherichia coli, Synechococcus elongatus, or Pichia pastoris yeast as metabolic factories for HMB production in the embodiments described above. Especially noteworthy is the fact that Escherichia coli can achieve high yields of HMB using only the minimal M9 basal medium with pure salts, showcasing significant potential for industrial application.

Currently, the price of glucose ranges from approximately $2.73 to $4 per kilogram, while the price of HMB ranges from about $20 to $30 per kilogram, with an estimated global market value of $90 million. The theoretical yield of HMB produced from glucose is 1.5 moles of glucose producing 1 mole of HMB, equivalent to 118.13 grams of HMB from 270 grams of glucose. Therefore, the theoretical yield is approximately 0.44 g/g of glucose. If the current cost of glucose is $2.73 per kilogram and the selling price of HMB is $20 per kilogram, the breakeven point is achieved when the yield of HMB exceeds or equals 0.1365 grams of HMB per gram of glucose. In the embodiments of the present invention, the highest HMB yield from Escherichia coli is up to 0.32 grams of HMB per gram of glucose, demonstrating significant commercial viability.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. A transgenic strain for producing 3-hydroxy-3-methylbutyric acid, comprising:
a host cell having a metabolic pathway for converting a carbon source into acetyl-CoA, wherein the host cell is Escherichia coli, cyanobacteria, or yeast, and the carbon source comprises at least one of glucose, xylose, acetic acid or acetate, methanol, and carbon dioxide; and
a plurality of exogenous genes located in the host cell to assemble an anabolic pathway from acetyl-CoA (Ac-CoA) to 3-hydroxy-3-methylbutyrate (HMB), wherein the exogenous genes comprise:
a first exogenous gene, which is a gene encoding acetyl-CoA acetyltransferase (AtoB) or acetyl-CoA thiolase (NphT7), or a gene with 80% or more sequence identity to SEQ ID NO: 1 or SEQ ID NO: 2 and possessing AtoB activity or NphT7 activity;
a second exogenous gene, which is a gene encoding hydroxymethylglutaryl-CoA synthase (MvaS), or a gene with 70% or more sequence identity to SEQ ID NO: 3 and possessing MvaS activity;
a third exogenous gene, which is a gene encoding 3-hydroxy-3-methylglutaryl CoA dehydratase (LiuC), or gene with 50% or more sequence identity to SEQ ID NO: 4 and possessing LiuC activity;
a fourth exogenous gene, which is a gene encoding 3-methylglutaryl-CoA decarboxylase (AibAB), or a gene with 70% or more sequence identity to SEQ ID NO: 5 and SEQ ID NO: 6 and possessing AibAB activity when expressed and combined; and
a fifth exogenous gene, which is a gene encoding a thioester hydrolase (YciA, TesB, MenI, or YqiA), or a gene with 60% or more sequence identity to SEQ ID NO: 7, SEQ ID NO: 8, SEQ ID NO: 9, or SEQ ID NO: 10, and possessing YciA, TesB, MenI, or YqiA activity when expressed.

2. The transgenic strain of claim 1, further comprising at least a recombinant plasmid carrying the exogenous genes.

3. The transgenic strain of claim 1, wherein the exogenous genes are integrated into a genome of the host cell.

4. The transgenic strain of claim 1, wherein the Escherichia coli comprises a plurality of substrains directly utilizing exogenous carbon sources to synthesize acetyl-CoA in vitro, and the Escherichia coli substrains comprise BW25113, XL1-Blue, DH5a, W3110, NEBExpress®, Rosetta (DE3), W strain, crooks, and the probiotic E. coli Nissle 1917.

5. The transgenic strain of claim 1, wherein the Escherichia coli has gene deletions of metabolic pathways competing with the anabolic pathway of 3-hydroxy-3-methylbutyratethe.

6. The transgenic strain of claim 5, wherein a gene of the Escherichia coli encoding pyruvate oxidase (PoxB) is knockout to reduce the production of acetate byproducts in the pathway synthesizing acetate from pyruvate.

7. The transgenic strain of claim 5, wherein a gene of the Escherichia coli encoding citrate synthase (GltA) in the TCA cycle is knockout to increase the yield of 3-hydroxy-3-methylbutyrate as the main product.

8. The transgenic strain of claim 1, wherein the cyanobacteria comprise Synechococcus elongatus.

9. The transgenic strain of claim 1, wherein the yeast comprises Pichia pastoris.

10. A method for producing 3-hydroxy-3-methylbutyrate, comprising:
culturing the transgenic strains of claim 1 to allow the transgenic strain to produce 3-hydroxy-3-methylbutyrate, wherein culturing conditions comprise a first culturing temperature before inducing the expression of the exogenous genes in the transgenic strain is 37±2° C., a second culturing temperature after inducing the expression of the exogenous genes in the transgenic strain is 30±2° C., and a dissolved oxygen level in a bacterial solution of the transgenic strain is 30%.

11. The method of claim 10, wherein a culture medium used is M9 minimal medium when the transgenic strain is the Escherichia coli.

12. The method of claim 10, wherein a culture medium used is modified BG-11 medium containing 50±10 mM NaHCO₃ when the transgenic strain is the cyanobacteria.

13. The method of claim 10, wherein a culture medium used is YPB complex medium when the transgenic strain is the yeast.


SEQUENCE LISTING (13 sequences)

SEQ ID NO: 1 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 2 - protein, 329 aa, Streptomyces sp.
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 3 - protein, 388 aa, Staphylococcus aureus
MTIGIDKINFYVPKYYVDMAKLAEARQVDPNKFLIGIGQTEMAVSPVNQDIVSMGANAAKDIITDEDKKKIGMVIVATESAVDAAKAAAVQIHNLLGIQPFARCFEMKEACYAATPAIQLAKDYLATRPNEKVLVIATDTARYGLNSGGEPTQGAGAVAMVIAHNPSILALNEDAVAYTEDVYDFWRPTGHKYPLVDGALSKDAYIRSFQQSWNEYAKRQGKSLADFASLCFHVPFTKMGKKALESIIDNADETTQERLRSGYEDAVDYNRYVGNIYTGSLYLSLISLLENRDLQAGETIGLFSYGSGSVGEFYSATLVEGYKDHLDQAAHKALLNNRTEVSVDAYETFFKRFDDVEFDEEQDAVHEDRHIFYLSNIENNVREYHRPE

SEQ ID NO: 4 - protein, 258 aa, Stigmatella aurantiaca
MPEFKVDARGAIEIWTIDGADRRNAISRAMLQELSGMVTRVSTGRAVRAVIITGAGDKAFCAGADLKERAGMSEAEVRAFLDGLRQTLRAIEKSDCVFIAAINGAALGGGTELSLACDLRVAVPATELGLTEVRLGIIPGGGGTQRLSRLVGPGRAKDLILTGRRINAAEAFSIGLVNRLAPEGHLVETSFSLAEAIVANAPIAVSTAKHAIDEGTGLELDDALALELRKYEDILQTEDRLEGLRSFAEKRPPVYKGR

SEQ ID NO: 5 - protein, 266 aa, Stigmatella aurantiaca
MRFHGWRPLSEAVASISDGAWLATGGFMLGRAPMALVLELIAQKRQGLRLISLPNPLPAEFLVAGGCLAHVDLPFGALNLEGRVRPMPCLKRAIEQNRLSWREHDGYRVVQRLRAASMGLPFLPAPDTDVSALASAEPPRTVVDPFTGQTVTVEPAFYPDVALVHAQAADERGNLYIEDPTTDLLVAGAARRVIATAEQRVPRLERVTVPGFQVESVSLAPRGALPTGCLGLYAHDDAMLAHYLELAEAGREAEFLSRLLDARRAA

SEQ ID NO: 6 - protein, 246 aa, Stigmatella aurantiaca
MSTPSEATPAETVVALLAREIEDGAVIATGVASPLAILAIAVARATHAPRLTYLACVGSLDPALPTLLPSSEDLGYLLGRTAEITIADLFDHARRGRVDTIFFGAAEVDARGRTNMTAAGSLERPRVKFPGVAGAATLRQWVRRPVLLVPKQSRRNLVPEVQVATTQDPRRPVRLISDLGIFELGAEGARLHARHAWATAADISERTGFSFSVAAPLPVTPPPDARTLEAIRTIDSHCFRDQLVGA

SEQ ID NO: 7 - protein, 132 aa, Escherichia coli
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 8 - protein, 286 aa, Escherichia coli
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 9 - protein, 136 aa, Escherichia coli
MIWKRKITLEALNAMGEGNMVGFLDIRFEHIGDDTLEATMPVDSRTKQPFGLLHGGASVVLAESIGSVAGYLCTEGEQKVVGLEINANHVRSAREGRVRGVCKPLHLGSRHQVWQIEIFDEKGRLCCSSRLTTAIL

SEQ ID NO: 10 - protein, 193 aa, Escherichia coli
MSTLLYLHGFNSSPRSAKASLLKNWLAEHHPDVEMIIPQLPPYPSDAAELLESIVLEHGGDSLGIVGSSLGGYYATWLSQCFMLPAVVVNPAVRPFELLTDYLGQNENPYTGQQYVLESRHIYDLKVMQIDPLEAPDLIWLLQQTGDEVLDYRQAVAYYASCRQTVIEGGNHAFTGFEDYFNPIVDFLGLHHL

SEQ ID NO: 11 - protein, 258 aa, Myxococcus xanthus
MPEFKVDARGPIEIWTIDGESRRNAISRAMLKELGELVTRVSSSRDVRAVVITGAGDKAFCAGADLKERATMAEDEVRAFLDGLRRTFRAIEKSDCVFIAAINGAALGGGTELALACDLRVAAPAAELGLTEVKLGIIPGGGGTQRLARLVGPGRAKDLILTARRINAAEAFSVGLANRLAPEGHLLAVAYGLAESVVENAPIAVATAKHAIDEGTGLELDDALALELRKYEEILKTEDRLEGLRAFAEKRAPVYKGR

SEQ ID NO: 12 - protein, 265 aa, Myxococcus xanthus
MKTARWCSLEEAVASIPDGASLATGGFMLGRAPMALVMELIAQGKRDLGLISLPNPLPAEFLVAGGCLARLEIAFGALSLQGRVRPMPCLKRAMEQGTLAWREHDGYRVVQRLRAASMGLPFIPAPDADVSGLARTEPPPTVEDPFTGLRVAVEPAFYPDVALLHARAADERGNLYMEDPTTDLLVAGAAKRVIATVEERVAKLPRATLPGFQVDRIVLAPGGALPTGCAGLYPHDDEMLARYLSLAETGREAEFLETLLTRRAA

SEQ ID NO: 13 - protein, 246 aa, Myxococcus xanthus
MSATLDITPAETVVSLLARQIDDGGVVATGVASPLAILAIAVARATHAPDLTYLACVGSLDPEIPTLLPSSEDLGYLDGRSAEITIPDLFDHARRGRVDTVFFGAAEVDAEGRTNMTASGSLDKPRTKFPGVAGAATLRQWVRRPVLLVPRQSRRNLVPEVQVATTRDPRRPVTLISDLGVFELGASGARLLARHPWASEEHIAERTGFAFQVSEALSVTSLPDARTVAAIRAIDPHGYRDALVGA